(19) United States
(12) Patent Application Publication
(10) Pub. No.: US 2026/0215146 A1
(43) Pub. Date: Jul. 23, 2026

(54) ORGANIC SEMICONDUCTOR POLYMER, ORGANIC THIN FILM TRANSISTOR, STRETCHABLE PANEL, AND ELECTRONIC DEVICE

(71) Applicant: Samsung Electronics Co., Ltd., Suwon-si (KR)

(72) Inventors: Seon-Jeong LIM, Suwon-si (KR); Jiyoung JUNG, Suwon-si (KR); Kyunghun KIM, Suwon-si (KR); Bang Lin LEE, Suwon-si (KR); Gae Hwang LEE, Suwon-si (KR); Hyung Jun KIM, Suwon-si (KR); Hyun Bum KANG, Suwon-si (KR)

(73) Assignee: Samsung Electronics Co., Ltd., Suwon-si (KR)

(21) Appl. No.: 19/455,187

(22) Filed: Jan. 21, 2026

(30) Foreign Application Priority Data
Jan. 22, 2025 (KR) 10-2025-0009846

(51) Int. Cl. H10K 85/10; A61B 5/00; H10H 29/32; H10K 10/46; H10K 19/10; H10K 59/125; H10K 77/10

(52) U.S. Cl. CPC H10K 85/113; H10H 29/32; H10K 10/466; H10K 10/484; H10K 19/10; H10K 59/125; H10K 77/111; A61B 5/6824; A61B 5/6825; A61B 2562/164

(57) ABSTRACT

Disclosed are an organic semiconductor polymer including a structural unit represented by Chemical Formula 1, an organic thin film transistor, and an electronic device including the organic semiconductor polymer:

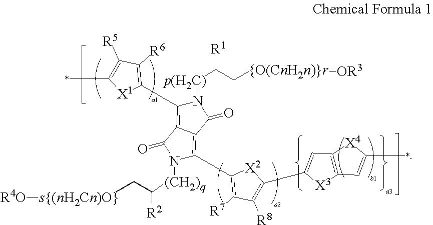

### CROSS-REFERENCE TO RELATED APPLICATION

This application claims priority to and the benefit of Korean Patent Application No. 10-2025-0009846 filed in the Korean Intellectual Property Office on Jan. 22, 2025, the entire contents of which are incorporated herein by reference.

### BACKGROUND

### 1. Field

An organic semiconductor polymer, an organic thin film transistor, a stretchable panel, and an electronic device are related.

### 2. Description of the Related Art

Flat panel displays, such as liquid crystal displays (LCD), organic light emitting diode (OLED) displays, electrophoretic displays or the like, may include multiple pairs of field generating electrodes and an electro-optical active layer interposed therebetween. For example, LCDs may include an electro-optical active layer of a liquid crystal layer, and OLED displays may include an electro-optical active layer of an organic emission layer.

One of the paired field generating electrodes may be generally connected to a switching element and applied with an electrical signal, and the electro-optical active layer may be configured to transform the electrical signal to an optical signal to display an image.

The flat panel display may include a three-terminal element such as a thin film transistor (TFT) as the switching element, and may also include a gate line configured to transfer a scan signal for controlling the thin film transistor and a data line configured to transfer a data signal to be applied to a pixel electrode.

Organic thin film transistor (OTFT) may be shaped in a fiber or a film form according to the organic material characteristic, and so has drawn attention as a core element for a flexible display device. The organic thin film transistor may be manufactured using a solution process, e.g., inkjet printing, and may be more easily applied to a large area flat panel display where a deposition process has a limit. Therefore, OTFT, including an organic semiconductor, e.g., a low molecule or a polymer, instead of the inorganic semiconductor, e.g., silicon (Si), have been researched.

### SUMMARY

Example embodiments provide an organic semiconductor polymer having improved charge mobility and being applicable to manufacturing organic transistors using a solution process.

Example embodiments also provide an organic thin film transistor including the organic semiconductor polymer.

Example embodiments also provide an electronic device including the organic semiconductor polymer or the organic thin film transistor.

Example embodiments also provide a stretchable panel including a stretchable thin film including the organic semiconductor polymer.

Example embodiments also provide an electronic device including the stretchable panel.

According to some example embodiments, an organic semiconductor polymer includes a structural unit represented by Chemical Formula 1:

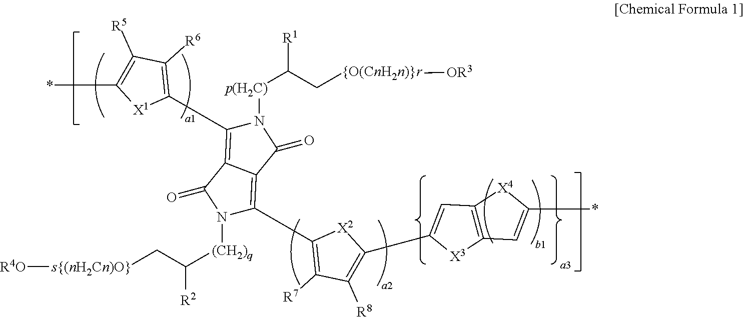

In Chemical Formula 1,
R¹, R², R³, and R⁴ are each independently a substituted or unsubstituted C1 to C30 alkyl group,
R⁵, R⁶, R⁷, and R⁸ are each independently hydrogen, a substituted or unsubstituted C1 to C30 alkyl group, a substituted or unsubstituted C3 to C30 cycloalkyl group, a substituted or unsubstituted C6 to C30 aryl group, a substituted or unsubstituted C7 to C30 arylalkyl group, a substituted or unsubstituted C1 to C30 heteroalkyl group, a substituted or unsubstituted C2 to C30 heterocycloalkyl group, a substituted or unsubstituted C2 to C30 alkenyl group, a substituted or unsubstituted C2 to C30 alkynyl group, a hydroxy group, a halogen atom, or a combination thereof,
X¹, X², X³, and X⁴ are each independently S, Se, or Te,
a1 and a2 are each independently an integer from 1 to 5,
a3 is an integer from 0 to 5,
b1 is 0 or 1,
n is an integer from 2 to 5,
p and q are each independently an integer from 1 to 20, and
r and s are each independently an integer from 1 to 20.

In at least one embodiment, R¹, R², R³, and R⁴ are each independently a substituted or unsubstituted C1 to C20 alkyl group, R⁵, R⁶, R⁷, and R⁸ are each independently hydrogen, a substituted or unsubstituted C1 to C20 alkyl group, a substituted or unsubstituted C3 to C20 cycloalkyl group, a substituted or unsubstituted C6 to C20 aryl group, a substituted or unsubstituted C7 to C20 arylalkyl group, a substituted or unsubstituted C1 to C20 heteroalkyl group, a substituted or unsubstituted C2 to C20 heterocycloalkyl group, a substituted or unsubstituted C2 to C20 alkenyl group, a substituted or unsubstituted C2 to C20 alkynyl group, a hydroxy group, a halogen atom, or a combination thereof, X¹, X², X³, and X⁴ are each independently S, or Se, a1 and a2 are each independently from 1 to 3, a3 is from 0 to 3,
b1 0 or 1,
n is of 2 or 3,
p and q are each independently an integer of from 1 to 10, and
r and s are each independently an integer of from 1 to 10.

In at least one embodiment,
R¹ and R², are each independently a substituted or unsubstituted C5 to C20 alkyl group,
R³ and R⁴ are each independently a substituted or unsubstituted C1 to C5 alkyl group,
p and q are each independently an integer from 2 to 10, and
r and s are each independently an integer from 1 to 5.

In at least one embodiment,
R⁵, R⁶, R⁷, and R⁸ are each independently hydrogen, a substituted or unsubstituted C1 to C10 alkyl group, a substituted or unsubstituted C3 to C10 cycloalkyl group, a substituted or unsubstituted C6 to C10 aryl group, a substituted or unsubstituted C7 to C10 arylalkyl group, a substituted or unsubstituted C1 to C10 heteroalkyl group, a substituted or unsubstituted C2 to C10 heterocycloalkyl group, a substituted or unsubstituted C2 to C10 alkenyl group, a substituted or unsubstituted C2 to C10 alkynyl group, a hydroxy group, a halogen atom, or a combination thereof,
X¹ and X² are each independently S, and
X³ and X⁴ are each independently S or Se.

In at least one embodiment,
a1, a2, and a3 are each independently 1 or 2, and
b1 is 0 or 1.
at least one embodiment, Chemical Formula 1 may be represented by at least one of Chemical Formula 2, Chemical Formula 3, or Chemical Formula 3:

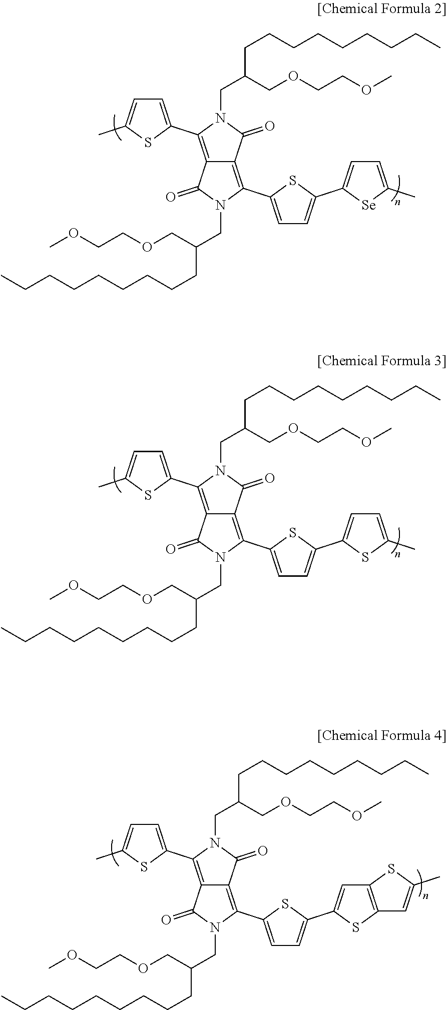

According to some example embodiments, an organic thin film transistor includes a gate electrode, an organic semiconductor overlapping the gate electrode, a gate insulating layer separating the gate electrode from the organic semiconductor, and a source electrode and a drain electrode electrically connected to the organic semiconductor, wherein the organic semiconductor includes an organic semiconductor polymer including a structural unit represented by the Chemical Formula 1.

The organic thin film transistor may be configured as a stretchable thin film transistor.

The gate electrode, the source electrode, and the drain electrode may each independently include a microcrack metal, a liquid metal, a conductive nanostructure, a conductive polymer, or a combination thereof, and the gate insulating layer may include polyorganosiloxane, a polymer including a butadiene structural unit, a polymer including an olefin structural unit, a polymer including a urethane structural unit, a polymer including an acrylic structural unit, or any combination thereof.

According to some example embodiments, an electronic device includes the organic thin film transistor of example embodiments.

The electronic device may include a display device, and an organic sensor.

According to some example embodiments, an electronic device includes the organic semiconductor polymer of example embodiments in the active layer.

The electronic device may include a solar cell, a display device, or an organic sensor.

According to some example embodiments, a stretchable panel includes a stretchable substrate, a stretchable thin film transistor array arranged on the stretchable substrate and including a plurality of stretchable thin film transistors, and a unit element array including a plurality of unit elements electrically connected to each of the plurality of stretchable thin film transistors.

The stretchable panel may further include a non-stretchable pattern overlapping with a portion of the stretchable substrate and having a higher elastic modulus than the stretchable substrate, and the stretchable panel may include a high elastic modulus region in which the non-stretchable pattern is formed, and a low elastic modulus region excluding the high elastic modulus region.

The stretchable substrate may include at least one of a polyorganosiloxane, a polymer including a butadiene structural unit, a polymer including an olefin structural unit, a polymer including a urethane structural unit, a polymer including an acrylic structural unit, or a combination thereof, and the non-stretchable pattern may include at least one of a polycarbonate, a polymethylmethacrylate, a polyethylene terephthalate, a polyethylene naphthalate, a polyimide, a polyamide, a polyamideimide, a polyethersulfone, or a combination thereof.

Each of the plurality of unit elements may include a light emitting diode, a photoelectric conversion diode, or a combination thereof, and the plurality of unit elements may be in the high elastic modulus region.

The stretchable panel may be at least one of a stretchable display panel or a stretchable sensor array.

According to some example embodiments, an electronic device including the stretchable panel is provided.

The organic semiconductor polymer of some example embodiments has a comparatively high electron mobility and comparatively excellent solubility. Accordingly, the organic semiconductor polymer may be applied by a solution process, allowing easily manufacturing an organic thin film transistor, or an electronic device including the organic thin film transistor. Thus, fabricated organic thin film transistor or electronic device may have a high electron mobility, and exhibit excellent electronic properties.

### BRIEF DESCRIPTION OF THE DRAWINGS

FIG. 1 is a cross-sectional view of an organic thin film transistor according to some example embodiments,

FIG. 2 is a perspective view showing an example of a stretchable thin film transistor according to some example embodiments,

FIG. 3 is a plan view showing an example of a stretchable panel according to some example embodiments,

FIG. 4 is a cross-sectional view showing an example of a unit element,

FIG. 5 is a plan view showing another example of a stretchable panel according to some example embodiments,

FIG. 6 is a schematic diagram showing a stretchable display panel according to some examples,

FIGS. 7A, 7B, and 7C are schematic views showing sensor arrays according to some examples,

FIG. 8 is a ¹H-NMR spectrum of the acceptor compound prepared in Synthesis Example 1,

FIG. 9 is a graph showing light absorbing characteristics of the organic polymer thin films according to Examples 1 to 3 and Comparative Example 1,

FIG. 10 are surface images of the organic polymer thin film according to Example 3, taken by atomic force microscopy (AFM),

FIG. 11 are surface images of the organic polymer thin film according to Comparative Example 1, taken by atomic force microscopy (AFM),

FIG. 12 is a graph showing the current characteristics of the organic thin film transistor according to Example 1,

FIG. 13 is a graph showing the current characteristics of the organic thin film transistor according to Example 2,

FIG. 14 is a graph showing the current characteristics of the organic thin film transistor according to Example 3, and

FIG. 15 is a graph showing the current characteristics of the organic thin film transistor according to Comparative Example 1.

### DETAILED DESCRIPTION

Hereinafter, some example embodiments will be described in detail so that those of ordinary skill in the art may easily implement them. However, actually applied structures may be implemented in several different forms and are not limited to the embodiments described herein.

In the drawings, the thickness of layers, films, panels, regions, etc., may be exaggerated for clarity.

In the present specification, a horizontal direction may include a first horizontal direction (X direction) and a second horizontal direction (Y direction) that intersect each other. A direction intersecting the first horizontal direction (X direction) and the second horizontal direction (Y direction) may be referred to as a vertical direction (Z direction). In the present specification, a vertical level may be referred to as a height level according to a vertical direction (Z direction) of an arbitrary configuration. In other words, it will be understood that the spatially relative terms are intended to encompass different orientations of the device in use, operation, and/or manufacture, in addition to the orientation depicted in the figures. For example, if the device in one of the figures is turned over, an element or feature described to be “below” another element or feature would then be oriented “above” the other element or feature.

Additionally, when the terms “about” or “substantially” are used in this specification in connection with a numerical value and/or geometric term, it is intended that the associated numerical value includes a manufacturing tolerance (e.g., ±10%) around the stated numerical value. Further, regardless of whether numerical values and/or geometric terms are modified as “about” or “substantially,” it will be understood that these values should be construed as including a manufacturing or operational tolerance (e.g., ±10%) around the stated numerical values and/or geometry. Further, ranges of and/or from “A to B” include both “A” and “B”, unless indicated otherwise.

It will be understood that when an element such as a layer, film, region, or substrate is referred to as being “on” another element, it may be directly on the other element or intervening elements may also be present. In contrast, when an element is referred to as being “directly on” another element, there are no intervening elements present.

As used herein, when a definition is not otherwise provided, “substituted” refers to replacement of hydrogen of a compound or a functional group by a substituent selected from deuterium, a halogen atom, a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, sulfonic acid group or a salt thereof, a phosphoric acid group or a salt thereof, a silyl group, a C1 to C20 alkyl group, a C2 to C20 alkenyl group, a C2 to C20 alkynyl group, a C6 to C30 aryl group, C7 to C30 arylalkyl group, C1 to C30 alkoxy group, a C1 to C20 heteroalkyl group, a C3 to C20 heteroaryl group, C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and/or any combination thereof.

Hereinafter, “polymer” includes a homopolymer, a copolymer, and/or any combination thereof.

Hereinafter, “combination” includes a mixture, a composite, and/or a stacked structure of two or more layers.

Hereinafter, when a definition is not otherwise provided, the term “hetero” refers to one including 1 to 3 heteroatoms selected from N, O, S, Se, and/or P.

One known method for improving the hole charge mobility of organic polymer semiconductor materials is to increase the crystallinity of the polymer thin film. Strong polymer-polymer interactions (inter-chain interactions) are a component for achieving high crystallinity. In order to achieve this, molecules may be designed to have a high degree of planarity in the polymer backbone. Furthermore, the structure of the side chain of the polymer may be modified to improve polymer solubility, and the side chain structure may significantly affect the crystallinity of the polymer. For example, linear side chains may be preferred for high polymer crystallinity. However, if crystallinity becomes too high, the polymer's solubility decreases. This low solubility may make it difficult to manufacture thin film transistors (TFTs) using solution processes. Therefore, a method of introducing branched alkyl groups into the side chains has been proposed to increase polymer solubility.

However, although the solubility of the polymer may improve by introducing a branched alkyl side chain, making it possible to manufacture TFTs through a solution process, the branched alkyl group has a bulky structural characteristic compared to a linear alkyl group, which hinders polymer-polymer interaction (inter-chain interaction), limiting the implementation of the maximum crystallinity that a polymer thin film may have. As an approach to address these issues, a polymer structure comprising a branched ethylene glycol (EG) side chain, which may replace the rigid branched alkyl group, has been proposed. In this case, the flexible EG side chain structure may induce stronger polymer-polymer backbone interactions. For this purpose, a diketopyrrolopyrrole (DPP)-based polymer material with a donor-acceptor structure composed only of branched EG side chains was proposed.

The hole charge mobility enhancement characteristic of the DPP polymer semiconductor material with a donor-acceptor structure composed only of branched EG side chains was reported in a paper (Chen et al., Angewandte Chemie, International Edition 2016, 55 (35), 10376-10380) and a patent document (CN115322339A), but when this was applied to an organic thin film transistor device, a problem was identified in that the transistor characteristics were not operated normally (see Comparative Example 1 herein). The inventors of the application have confirmed that a donor-acceptor type DPP-based polymer in which two branched side chains, one of which is an aliphatic alkyl group and the other is an alkylene glycol, for example, ethylene glycol (EG), are substituted at each nitrogen atom of DPP may have a high solubility in a solvent, and when a thin film prepared therefrom is applied to a transistor device, excellent transistor characteristics are obtained.

Accordingly, some embodiments provide an organic semiconductor polymer as a donor-acceptor polymer in which each nitrogen atom of DPP is substituted with a branched side chain, wherein one of the two branched chains is an aliphatic alkyl group and the other is an alkylene glycol.

The organic semiconductor polymer of some example embodiments may include a structural unit represented by Chemical Formula 1:

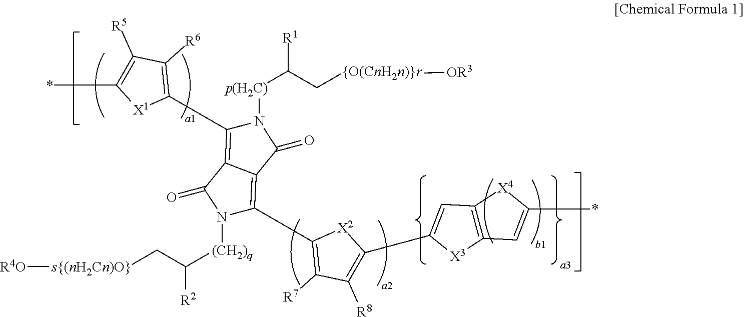

In Chemical Formula 1,
R¹, R², R³, and R⁴ may each independently be a substituted or unsubstituted C1 to C30 alkyl group,
R⁵, R⁶, R⁷, and R⁸ may each independently be hydrogen, a substituted or unsubstituted C1 to C30 alkyl group, a substituted or unsubstituted C3 to C30 cycloalkyl group, a substituted or unsubstituted C6 to C30 aryl group, a substituted or unsubstituted C7 to C30 arylalkyl group, a substituted or unsubstituted C1 to C30 heteroalkyl group, a substituted or unsubstituted C2 to C30 heterocycloalkyl group, a substituted or unsubstituted C2 to C30 alkenyl group, a substituted or unsubstituted C2 to C30 alkynyl group, a hydroxy group, a halogen atom, or a combination thereof,
X¹, X², X³, and X⁴ may each independently be S, Se, or Te,
a1 and a2 may each independently be an integer from 1 to 5, and
a3 is an integer from 0 to 5,
b1 is an integer of 0 or 1,
n is an integer from 2 to 5,
p and q may each independently be an integer from 1 to 20, and
r and s may each independently be an integer from 1 to 20.

In some embodiments, R¹, R², R³, and R⁴ may each independently be a substituted or unsubstituted C1 to C20 alkyl group.

In some embodiments, R⁵, R⁶, R⁷, and R⁸ may each independently be hydrogen, a substituted or unsubstituted C1 to C20 alkyl group, a substituted or unsubstituted C3 to C20 cycloalkyl group, a substituted or unsubstituted C6 to C20 aryl group, a substituted or unsubstituted C7 to C20 arylalkyl group, a substituted or unsubstituted C1 to C20 heteroalkyl group, a substituted or unsubstituted C2 to C20 heterocycloalkyl group, a substituted or unsubstituted C2 to C20 alkenyl group, a substituted or unsubstituted C2 to C20 alkynyl group, a hydroxy group, a halogen atom, or a combination thereof.

In some embodiments, X¹, X², X³, and X⁴ may each independently be S, or Se.

In some embodiments, a1 and a2 may each independently be an integer of from 1 to 3.

In some embodiments, a3 is an integer from 0 to 3.

In some embodiments, b1 is an inter of 0 or 1.

In some embodiments, n is an integer of 2 or 3.

In some embodiments, p and q may each independently be an integer from 1 to 10.

In some embodiments, r and s may each independently be an integer from 1 to 10.

In some embodiments, R¹ and R² may each independently be a substituted or unsubstituted C5 to C20 alkyl group, for example, a substituted or unsubstituted C8 to C15 alkyl group, a substituted or unsubstituted C10 to C15 alkyl group, or for example, a substituted or unsubstituted C10 to C12 alkyl group, but are not limited thereto.

For example, R¹ and R² may each independently be a substituted or unsubstituted C5 to C20 alkyl group, for example, a substituted or unsubstituted C8 to C15 alkyl group, a substituted or unsubstituted C10 to C15 alkyl group, or for example, a substituted or unsubstituted C10 to C12 alkyl group, but are not limited thereto.

For example, R¹ and R² may each independently be a substituted or unsubstituted C10 to C15 alkyl group, or for example, a substituted or unsubstituted C10 to C12 alkyl group, but are not limited thereto.

For example, R¹ and R² may be the same as or different from each other.

In some example embodiments, R³ and R⁴ may each independently be a substituted or unsubstituted C1 to C5 alkyl group, for example, a substituted or unsubstituted C1 to C4 alkyl group, a substituted or unsubstituted C1 to C3 alkyl group, a substituted or unsubstituted C1 to C2 alkyl group, or for example, a substituted or unsubstituted methyl group, but are not limited thereto.

For example, R³ and R⁴ may each independently be an unsubstituted pentyl group, unsubstituted buthyl group, unsubstituted propyl group, unsubstituted ethyl group, or for example, unsubstituted methyl group, but are not limited thereto.

For example, R³ and R⁴ may be the same as or different from each other.

In some example embodiments, r and s may each independently be an integer from 1 to 5, for example, from 1 to 4, from 1 to 3, from 1 or 2, or for example 1, but are not limited thereto.

For example, r and s may be the same as or different from each other.

In some example embodiments, p and q may each independently be an integer from 2 to 10, for example, from 3 to 10, from 3 to 9, from 3 to 8, from 4 to 10, from 4 to 9, or from 4 to 8, but are not limited thereto.

For example, p and q may be the same as or different from each other.

In some example embodiments, n may be an integer of 2 or 3, for example, 2.

When R¹, R², R³, and R⁴ are each the same as defined above, and r, s, p, q, and n are the integers within the range defined above, the organic semiconductor polymer including a structural unit represented by Chemical Formula 1 according to some embodiments may have comparatively excellent solubility in a solvent, while having comparatively good crystallinity when being formed into a thin film. Accordingly, the organic semiconductor polymer may be easily applicable in a solution process, which may lead to produce a thin film having a good charge mobility.

In some embodiments, X¹ and X² may each independently be S, and X³ and X⁴ may each independently be S or Se. For example, both X³ and X⁴ may be S, or may be Se. Or, for example, one of X³ and X⁴ may be S, and the other may be Se.

In some embodiments, a1, a2, and a3 may each independently be an integer of 1 or 2, for example, all of a1, a2, and a3 may be an integer of 1, or for example, all of a1, a2, and a3 may be an integer of 2. Alternatively, a1 and a2 may each be an integer of 2, and a3 may be an integer of 1. Or, for example, a1 and a2 may each be an integer of 2, and a3 may be an integer of 2.

In some embodiments, R⁵, R⁶, R⁷, and R⁸ may each independently be hydrogen, a substituted or unsubstituted C1 to C10 alkyl group, a substituted or unsubstituted C3 to C10 cycloalkyl group, a substituted or unsubstituted C6 to C10 aryl group, a substituted or unsubstituted C7 to C10 arylalkyl group, a substituted or unsubstituted C1 to C10 heteroalkyl group, a substituted or unsubstituted C2 to C10 heterocycloalkyl group, a substituted or unsubstituted C2 to C10 alkenyl group, a substituted or unsubstituted C2 to C10 alkynyl group, a hydroxy group, a halogen atom, or a combination thereof, and are not limited thereto.

For example, R⁵, R⁶, R⁷, and R⁸ may each independently be hydrogen, a substituted or unsubstituted C1 to C5 alkyl group, a substituted or unsubstituted C3 to C6 cycloalkyl group, a substituted or unsubstituted phenyl group, a substituted or unsubstituted C7 to C10 arylalkyl group, a substituted or unsubstituted C1 to C5 heteroalkyl group, a substituted or unsubstituted C2 to C5 heterocycloalkyl group, a substituted or unsubstituted C2 to C5 alkenyl group, a substituted or unsubstituted C2 to C5 alkynyl group, a hydroxy group, a halogen atom, or a combination thereof, and are not limited thereto.

For example, R⁵, R⁶, R⁷, and R⁸ may each independently be hydrogen, an unsubstituted C1 to C5 alkyl group, unsubstituted C2 to C5 alkenyl group, unsubstituted phenyl group, unsubstituted thiophene group, unsubstituted thiazole group, unsubstituted pyridine group, hydroxy group, a halogen atom, or a combination thereof, and are not limited thereto.

In some example embodiments, all of R⁵, R⁶, R⁷, and R⁸ may be hydrogen.

In some embodiments, Chemical Formula 1 may be represented by at least one of Chemical Formula 2, Chemical Formula 3, or Chemical Formula 4, and is not limited thereto:

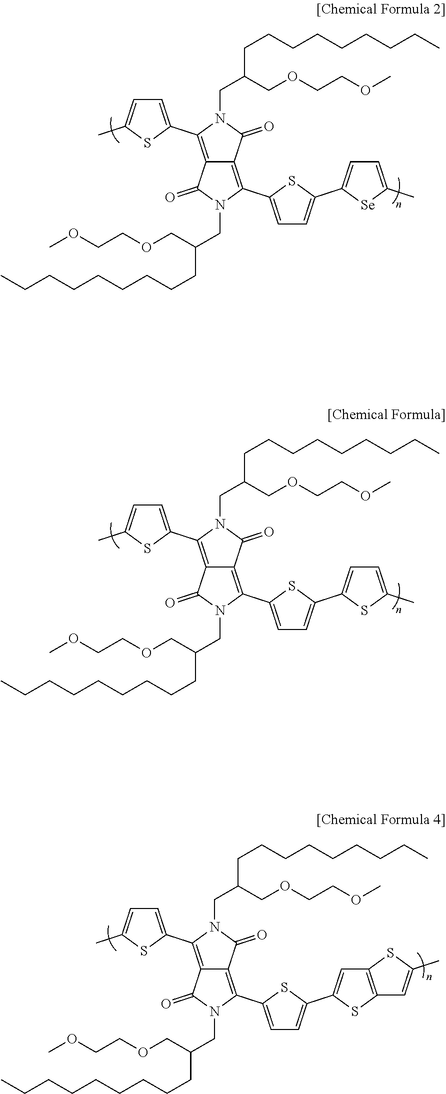

The organic semiconductor polymer including a structural unit represented by Chemical Formula 1 is polymer having bipolar characteristics by including a diketopyrrolopyrrole (DPP) moiety having n-type semiconductor characteristics, and moieties represented by a1, a2, and a3 having p-type semiconductor characteristics, such as, for example, a thiophene moiety when each X¹ to X³ is S, a selenophene moiety when each X¹ to X³ is Se, or a tellurophene moiety when each X¹ to X³ is Te, and thus, the organic semiconductor polymer may have a low energy band gap and be advantageous for intramolecular and intermolecular charge transfer. Accordingly, the organic semiconductor polymer may exhibit comparatively high charge mobility and comparatively low leakage current.

In addition, by possessing the branched side chain, the organic semiconductor polymer may have comparatively excellent solubility in an organic solvent, which is advantageous for solution processing, thereby having comparatively good processability and comparatively good properties for forming thin film. The organic semiconductor polymer may be applied to various devices including organic semiconductors and/or OTFT. For example, the organic semiconductor polymer may be applied to an organic thin film transistor and be applied as an active layer in an electronic device such as, for example, a solar cell, an organic light-emitting display, and an organic sensor.

According to some example embodiments, an organic thin film transistor includes a gate electrode, an organic semiconductor overlapping the gate electrode, and a source electrode and a drain electrode electrically connected to the organic semiconductor, wherein the organic semiconductor includes a structural unit represented by the Chemical Formula 1.

A weight average molecular weight of the organic semiconductor polymer may be from about 5,000 g/mol to about 1,000,000 g/mol, for example, from about 10,000 g/mol to about 1,000,000 g/mol, from about 10,000 g/mol to about 500,000 g/mol, from about 10,000 g/mol to about 300,000 g/mol, from about 10,000 g/mol to about 250,000 g/mol, from about 10,000 g/mol to about 200,000 g/mol, from about 20,000 g/mol to about 200,000 g/mol, from about 30,000 g/mol to about 200,000 g/mol, from about 30,000 g/mol to about 150,000 g/mol, from about 30,000 g/mol to about 120,000 g/mol, from about 50,000 g/mol to about 200,000 g/mol, from about 50,000 g/mol to about 150,000 g/mol, from about 50,000 g/mol to about 120,000 g/mol, from about 50,000 g/mol to about 100,000 g/mol, from about 80,000 g/mol to about 200,000 g/mol, from about 80,000 g/mol to about 150,000 g/mol, and/or, for example, from about 80,000 g/mol to about 120,000 g/mol, but is not limited to these ranges.

Hereinafter, an example of an organic thin film transistor including the organic semiconductor polymer is described referring to the drawing.

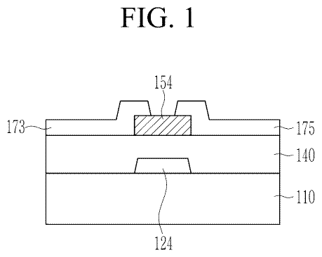

FIG. 1 is a cross-sectional view of an organic thin film transistor according to some example embodiments.

A gate electrode 124 may be formed on a substrate 110 made of transparent glass, silicon, or plastic. The gate electrode 124 may be connected to a gate line (not shown) configured to transmit a gate signal. The gate electrode 124 may be made of and/or include a conductive material, such as gold (Au), copper (Cu), nickel (Ni), aluminum (Al), molybdenum (Mo), chromium (Cr), tantalum (Ta), titanium (Ti), an alloy thereof, a combination thereof, and/or the like.

A gate insulating layer 140 may be formed on the gate electrode 124. The gate insulating layer 140 may be made of and/or include an organic insulating material or an inorganic insulting material. Examples of the organic insulating material may include a soluble polymer compound, e.g., a polyvinyl alcohol-based compound, a polyimide-based compound, a polyacryl-based compound, a polystyrene-based compound, benzocyclobutane (BCB), and/or the like; examples of the inorganic insulating material may include a silicon nitride (SiNx), silicon oxide (SiO₂), and/or the like.

A source electrode 173 and a drain electrode 175 may be formed on the gate insulating layer 140. The source electrode 173 and the drain electrode 175 may face each other with the gate electrode 124 therebetween. The source electrode 173 is electrically connected to the data line (not shown) configured to transfer the data signal. The source electrode 173 and the drain electrode 175 may include a conductive material, such as at least one metal selected from gold (Au), copper (Cu), nickel (Ni), aluminum (Al), molybdenum (Mo), chromium (Cr), tantalum (Ta), titanium (Ti), an alloy thereof, a combination thereof, and/or the like.

An organic semiconductor layer 154 may be formed on the source electrode 173 and the drain electrode 175. The organic semiconductor layer 154 may be made of the above organic semiconductor polymer. In other words, the organic semiconductor layer 154 may include the organic semiconductor polymer including the structural unit represented by Chemical Formula 1. The organic semiconductor layer 154 may be formed in a solution process, e.g., spin coating, slit coating, or inkjet printing by preparing the above organic semiconductor polymer as a solution.

Although the bottom gate structured organic thin film transistor is illustrated as an organic thin film transistor, it is not limited thereto, and it may be applied to other organic thin film transistors, e.g., a top gate structured organic thin film transistor.

The organic thin film transistor may be applied to a switch or driving element of various electronic devices, and the electronic device may be, for example, a liquid crystal display (LCD), an organic light emitting diode (OLED) display, an eletrophoretic display device, or an organic sensor.

The organic thin film transistor may include a stretchable thin film transistor.

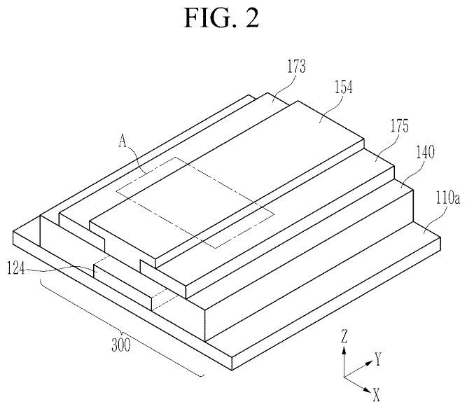

FIG. 2 is a perspective view showing an example of a stretchable thin film transistor according to some example embodiments.

Referring to FIG. 2, a stretchable thin film transistor 300 according to some example embodiments includes a gate electrode 124, a gate insulating layer 140, an organic semiconductor layer 154, a source electrode 173, and a drain electrode 175. The stretchable thin film transistor 300 may be supported by a supporting substrate, and the supporting substrate may be, for example, a stretchable substrate 110a.

The gate electrode 124 is electrically connected to a gate line (not shown) that transmits a gate signal and is overlapped with an organic semiconductor layer 154 and a gate insulating layer 140 to be described later. The gate electrode 124 may include, for example, a stretchable conductor. The stretchable conductor may include, but is not limited to, a metal (such as one or more of gold (Au), copper (Cu), nickel (Ni), aluminum (Al), molybdenum (Mo), chromium (Cr), tantalum (Ta), titanium (Ti), and/or an alloy thereof); a conductive nanostructure (such as a conductive nanowire or a conductive nanotube); a liquid metal; a conductive polymer; and/or a combination thereof. The metal may have a plurality of microcracks, for example microcracked Au configured to be stretchable (e.g., be configured to undergo non-plastic deform in one or more directions). The gate electrode 124 may be, for example, a stretchable electrode.

The gate insulating layer 140 may be disposed between the gate electrode 124 and the organic semiconductor layer 154 described later. The gate insulating layer 140 may be made of and/or include an organic insulator, an inorganic insulator, and/or an organic-inorganic insulator, and may include, for example, a stretchable insulator. The gate insulating layer 140 may include, for example, polyorganosiloxane, a polymer including a butadiene structural unit, a polymer including an olefin structural unit, a polymer including a urethane structural unit, a polymer including an acrylic structural unit, and/or a combination thereof. For example, the gate insulating layer 140 may include polydimethylsiloxane (PDMS), styrene-ethylene-butylene-styrene (SEBS), styrene-ethylene-propylene-styrene (SEPS), styrene-butadiene-styrene (SBS), styrene-isobutylene-styrene (SIBS), and/or any combination thereof, but the examples are not limited thereto. The gate insulating layer 140 may have, for example, one layer or two or more layers.

The source electrode 173 is electrically connected to a data line (not shown) that is configured to transmit a data signal and faces the drain electrode 175 with an organic semiconductor layer 154 described later therebetween. The source electrode 173 and the drain electrode 175 may be electrically connected to the organic semiconductor layer 154.

The source electrode 173 and the drain electrode 175 may include, for example, a stretchable conductor. The stretchable conductor may include, but is not limited to, a metal (such as gold (Au), copper (Cu), nickel (Ni), aluminum (Al), molybdenum (Mo), chromium (Cr), tantalum (Ta), titanium (Ti), and/or an alloy thereof); a conductive nanostructure (such as a conductive nanowire or a conductive nanotube); a liquid metal; a conductive polymer; or a combination thereof. The metal may have a plurality of microcracks, for example microcracked Au. The source electrode 173 and the drain electrode 175 may each be, for example, stretchable electrodes.

The organic semiconductor layer 154 may be disposed to be overlapped with the gate electrode 124 and electrically connected to the source electrode 173 and the drain electrode 175, respectively. The organic semiconductor layer 154 may include the organic semiconductor polymer according to some embodiment. In other words, the organic semiconductor layer 154 may include the organic semiconductor polymer including the structural unit represented by Chemical Formula 1. The organic semiconductor layer 154 may be formed by preparing a solution containing the organic semiconductor polymer, and applying the solution in a solution process, such as, for example, a spin coating, slit coating, or inkjet printing.

The aforementioned stretchable thin film transistors 300 may be repeatedly arranged along rows and/or columns on a stretchable substrate 110a to form a stretchable thin film transistor array, and the stretchable thin film transistor array may be included in a stretchable panel.

A stretchable panel according to some example embodiments is described below.

A stretchable panel according to some example embodiments may include any panel having an array of unit elements including a plurality of unit elements that operate in an active matrix manner arranged on a stretchable substrate that is deformable by an external force, and may include, for example, a flexible display panel, a stretchable display panel, a flexible sensor array panel, a stretchable sensor array panel, and/or any combination thereof, having flexible and/or stretchable characteristics.

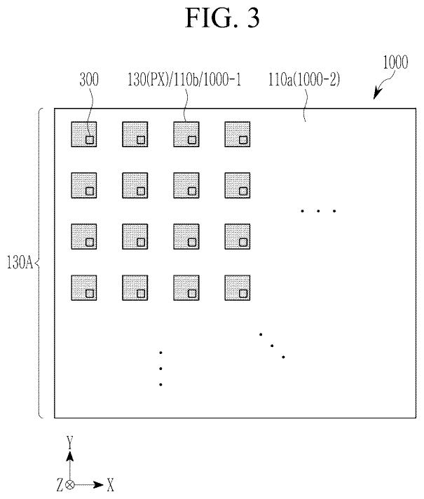

FIG. 3 is a plan view showing an example of a stretchable panel according to some example embodiments.

Referring to FIG. 3, a stretchable panel 1000 according to some example embodiments includes regions having different elastic moduli along an in-plane direction (e.g., X-Y direction) of a stretchable substrate 110a, and includes a high elastic modulus region 1000-1 having a relatively high elastic modulus and a low elastic modulus region 1000-2 having a relatively low elastic modulus.

The high elastic modulus region 1000-1 may be a region in which resistance to one or more external forces (such as twisting, pressing, pulling, etc.) is relatively high, so that the high elastic modulus region 1000-1 may not be substantially plastically deformed by the external force or a deformation degree may be very small. That is, the high elastic modulus region 1000-1 may include a stretch-resistant region with comparatively low stretchability due to a larger resistance to stretching, in addition to a region with no or very low stretchability at all.

The high elastic modulus region 1000-1 may be a region in which a non-stretchable pattern 110b having a high elastic modulus is covered on a stretchable substrate 110a, and accordingly, the high elastic modulus region 1000-1 may have substantially the same planar shape as the non-stretchable pattern 110b.

The elastic modulus of the high elastic modulus region 1000-1 may be determined by the elastic modulus of the non-stretchable pattern 110b. For example, the elastic modulus of the non-stretchable pattern 110b may be about 100 times or more, within the above range, about 300 times or more, about 500 times or more, or about 1000 times or more, and within the above range, about 100 times to about 108 times, about 500 times to about 108 times, about 1000 times to about 108 times, about 10 times to about 107 times, about 50 times to about 107 times, about 100 times to about 107 times, about 500 times to about 107 times, or about 103 times to about 107 times, higher than that of the stretchable substrate 110a. For example, the elastic modulus of the non-stretchable pattern 110b may be about 104 Pa to about 1012 Pa, but is not limited thereto. Due to the relatively high elastic modulus of the non-stretchable pattern 110b, the high elastic modulus region 1000-1 may not be substantially stretched or deformed even if the stretchable substrate 110a is stretched in a predetermined direction.

The non-stretchable pattern 110b may include an organic material, an inorganic material, an organic-inorganic material, and/or a combination thereof with a relatively high elastic modulus. For example, the non-stretchable pattern 110b may include polycarbonate, polymethylmethacrylate, polyethylene terephthalate, polyethylene naphthalate, polyimide, polyamide, polyamideimide, polyethersulfone, and/or a combination thereof, but the examples not limited thereto.

The non-stretchable pattern 110b may be formed by, for example, coating or depositing a material (e.g., an organic material) with a relatively high elastic modulus on the stretchable substrate 110a and partially removing it by, for example, etching, to leave the non-stretchable pattern 110b only in the portion corresponding to the high elastic modulus region 1000-1. However, the present disclosure is not limited thereto, and the high elastic modulus region 1000-1 and the low elastic modulus region 1000-2 having different elastic moduli may be implemented by forming the non-stretchable pattern 110b on the stretchable substrate 110a in various ways.

The high elastic modulus region 1000-1 may be and/or include a plurality of regions arranged, for example, along rows and/or columns, and the unit elements 130 described below may be arranged therein.

The low elastic modulus region 1000-2 is a region that may flexibly respond to external forces such as twisting, pressing, and pulling, and may be a region excluding the high elastic modulus region 1000-1. The low elastic modulus region 1000-2 may be a region where the non-stretchable pattern 110b is not covered on the stretchable substrate 110a and may be relatively evenly arranged on the entire surface of the stretchable panel 1000.

The elastic modulus of the low elastic modulus region 1000-2 may be substantially equal to the elastic modulus of the stretchable substrate 110a. The stretchable substrate 110a may include an elastomer with a relatively low elastic modulus, for example, an elastomer (including organic and inorganic elastomer), an inorganic elastomer-like material, or any combination thereof.

The elastomer may include for example polyorganosiloxane, a polymer including a butadiene structural unit, a polymer including an olefin structural unit, a polymer including a urethane structural unit, a polymer including an acrylic structural unit, or any combination thereof, for example polydimethylsiloxane, thermoplastic polyurethane (TPU), a styrene-ethylene-butylene-styrene (SEBS), styrene-ethylene-propylene-styrene (SEPS), styrene-butadiene-styrene (SBS), styrene-isoprene-styrene (SIS), styrene-isobutyrene-styrene (SIBS), and/or any combination thereof, but is not limited thereto. The inorganic elastomer-like material may include, for example, but not limited to, a ceramic having elasticity, a solid metal, a liquid metal, or any combination thereof. An elastic modulus of the elastomer may be, for example, about 100 Pa to about 109 Pa, but is not limited thereto.

The low elastic modulus region 1000-2 may be surrounded and isolated by the high elastic modulus region 1000-1, but is not limited thereto. Conversely, the high elastic modulus region 1000-1 may be surrounded and isolated by the low elastic modulus region 1000-2.

In the high elastic modulus region 1000-1, a plurality of unit elements 130 are arranged, and the plurality of unit elements 130 may be arranged along rows and/or columns to form a unit element array 130A.

Each unit element 130 may be, for example, a light emitting diode such as an organic light emitting diode, an inorganic light emitting diode, a quantum dot light emitting diode, a micro light emitting diode, a perovskite light emitting diode, a photoelectric conversion diode, an organic photoelectric conversion diode, an inorganic photoelectric conversion diode, an organic/inorganic photoelectric conversion diode, and/or any combination thereof, and may be the same or different from each other.

As an example, each unit element 130 may be a light emitting diode (LED) configured to, independently, emit light of a red spectrum, a green spectrum, a blue spectrum, and/or a combination thereof, but is not limited thereto. For example, in at least one embodiment, the unit elements 130 may be configured to emit light of a magenta spectrum, a yellow spectrum, and/or a cyan spectrum.

As an example, each unit element 130 may be a photoelectric conversion diode configured to selectively absorb red, green, blue, infrared, or a combination of light and convert the absorbed light into an electrical signal.

As an example, a portion of the unit element 130 may be a light emitting diode and a portion of the unit element 130 may be a photoelectric conversion diode.

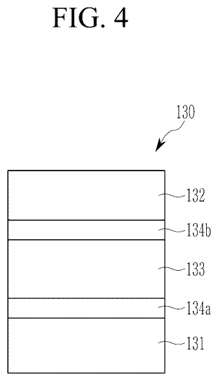

FIG. 4 is a cross-sectional view showing an example of a unit element.

Referring to FIG. 4, the unit element 130 may be a light emitting diode or a photoelectric conversion diode and may include an anode 131; a cathode 132; and an active layer 133 between the anode 131 and the cathode 132. In some embodiments, the unit element 130 may optionally include auxiliary layers 134a and 134b between the anode 131 and the active layer 133 and/or between the cathode 132 and the active layer 133.

At least one of the anode 131 or the cathode 132 may be a light transmitting electrode. For example, the anode 131 may be a light transmitting electrode and the cathode 132 may be a reflective electrode. For example, the anode 131 may be a reflective electrode and the cathode 132 may be a light transmitting electrode. For example, the anode 131 and the cathode 132 may each be a light transmitting electrode. At least one of the anode 131 or the cathode 132 may be a stretchable electrode. The stretchable electrode may include, for example, a stretchable conductor or may have a stretchable shape such as a wavy shape, a pleat shape, a pop-up shape, or a non-planar mesh shape. The stretchable electrode may have, for example, a plurality of microcracks, and since the plurality of microcracks are separated from each other like small holes, flexibility may be provided to the stretchable electrode by extending along the stretching direction (e.g., while be stretched) while maintaining the electrical movement path in the stretchable electrode.

The active layer 133 may be a light emitting layer or a photoelectric conversion layer.

The light emitting layer may be configured to emit light, e.g., in a red wavelength region, a green wavelength region, a blue wavelength region, an infrared wavelength region, and/or a combination thereof, and may include, for example, an organic light emitting layer, an inorganic light emitting layer (including a quantum dot light emitting layer), an organic/inorganic light emitting layer, or a combination thereof. The light emitting layer may include at least one host material and at least one dopant.

The photoelectric conversion layer may be configured to absorb light, e.g., in a red wavelength region, a green wavelength region, a blue wavelength region, an infrared wavelength region, and/or a combination thereof, and may be configured to convert the absorbed light into an electrical signal, and may be an organic photoelectric conversion layer, an inorganic photoelectric conversion layer, an organic/inorganic photoelectric conversion layer, or a combination thereof. The photoelectric conversion layer may include a p-type semiconductor and an n-type semiconductor, and the p-type semiconductor and the n-type semiconductor may form a p-n junction.

The auxiliary layers 134a and 134b may be, for example, charge auxiliary layers, and may be, for example, a hole transport layer, a hole injection layer, an electron blocking layer, an electron transport layer, an electron injection layer, a hole blocking layer, or a combination thereof, but are not limited thereto.

Each unit element 130 may be configured to be independently controlled and/or driven by one or more thin film transistors, at least some of which may be the aforementioned stretchable thin film transistors 300. For example, the stretchable thin film transistor 300 may be included in each pixel (subpixel) and may be arranged in a high elastic modulus region 1000-1. Each unit element 130 and the stretchable thin film transistor 300 may be electrically connected. The stretchable thin film transistor 300 may be, for example, the stretchable thin film transistor including the organic semiconductor layer 154 as described above.

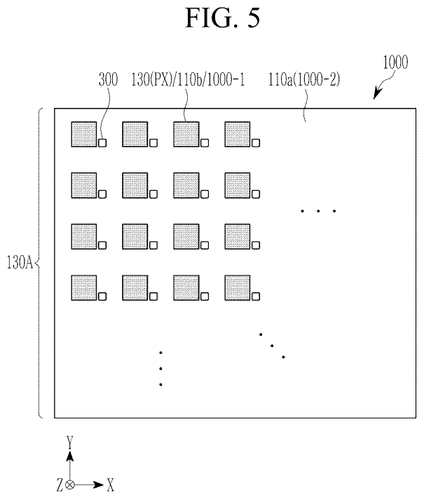

FIG. 5 is a plan view showing another example of a stretchable panel according to some example embodiments.

Referring to FIG. 5, the stretchable panel 1000 according to the present example includes a high elastic modulus region 1000-1 and a low elastic modulus region 1000-2, substantially similar to the above-described example, and the low elastic modulus region 1000-2 may be a region on the stretchable substrate 110a that is not covered with a non-stretchable pattern 110b, and the high elastic modulus region 1000-1 may be a region covered with a non-stretchable pattern 110b. Also, as in the aforementioned example, a plurality of unit elements 130 may be arranged in a high elastic modulus region 1000-1 to form a unit element array 130A, and each pixel (subpixel) may include a thin film transistor electrically connected to each unit element 130, and at least a portion of the thin film transistors may be the aforementioned stretchable thin film transistor 300.

However, unlike the aforementioned example, the stretchable panel 1000 according to the present example may have the stretchable thin film transistor 300 in the low elastic modulus region 1000-2. As described above, the stretchable thin film transistor 300 may include a stretchable electrode, a stretchable insulator, and a stretchable semiconductor layer, so that all components forming the stretchable thin film transistor 300 may be flexibly elongated by an external force and restored.

In this way, by including the aforementioned stretchable thin film transistor 300 as part of the thin film transistor and positioning the stretchable thin film transistor 300 in an area other than the pixel PX (low elastic modulus region 1000-2), the area occupied by the thin film transistor in the pixel PX may be reduced compared to a structure in which all thin film transistors are in each pixel PX.

Therefore, the space limitation of the pixels due to the low elastic modulus region 1000-2 for stretching may be overcome, the pixel size may be reduced, and the number of pixels per unit area may be increased accordingly. For example, the number of pixels per unit area in the stretchable panel 1000 may be greater than or equal to about 150 ppi (pixel per inch), greater than or equal to about 200 ppi, greater than or equal to about 250 ppi, greater than or equal to about 300 ppi, greater than or equal to about 350 ppi, greater than or equal to about 400 ppi, greater than or equal to about 450 ppi, or greater than or equal to about 500 ppi and may be, for example, about 150 ppi to about 1000 ppi, about 200 ppi to about 1000 ppi, about 250 ppi to about 1000 ppi, about 300 ppi to about 1000 ppi, about 350 ppi to about 1000 ppi, about 400 ppi to about 1000 ppi, about 450 ppi to about 1000 ppi, and/or about 500 ppi to about 1000 ppi.

The aforementioned stretchable panel 1000 may be applied to various fields requiring flexibility and/or stretchability, and may be, for example, a stretchable display panel or a stretchable sensor array. The stretchable panel 1000 may be, for example, a bendable display panel, a foldable display panel, a rollable display panel, a wearable device, a skin-type stretchable display panel, a skin-like display panel, a skin-like sensor array, a large-area conformable display, smart clothing, and/or the like, but is not limited thereto.

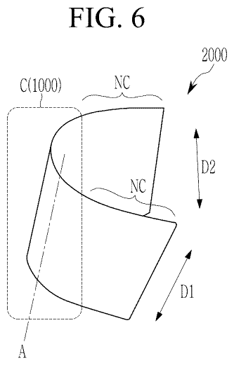

FIG. 6 is a schematic view showing a stretchable display panel according to some examples.

Referring to FIG. 6, the stretchable display panel 2000 according to some embodiments may be a bendable display panel capable of bending the screen along a predetermined direction. The stretchable display panel 2000 may be a display panel flexibly deformed by a user or an external force by introducing a structurally deformable portion into a screen for displaying an image. Herein, the structurally deformable portion may be at least a portion inside the screen.

The stretchable display panel 2000 may be bent along at least one axis A extending in the first direction D1. The stretchable display panel 2000 may include a deformation section (C) that is bent along an axis A and a non-deformable section NC excluding the deformable section C.

The deformable section C may be a bending section that is deformed into a curve around the axis A, and may be included in one or more of the stretchable display panel 2000. The deformable section C may be a region where a radius of curvature (which refers to a degree of being folded, bent, or rolled up to a maximum without substantial damage), is defined and where stress is concentrated, when repetitively folded, bent, or rolled.

The deformable section C of the stretchable display panel 2000 may include the stretchable panel 1000 including the high elastic modulus region 1000-1 and the low elastic modulus region 1000-2.

The high elastic modulus region 1000-1 is a region that is relatively resistant to external forces such as twisting, pressing, and pulling, and is substantially not deformed by the external force or is deformed to a very small degree, and unit elements 130 may be arranged in the high elastic modulus region 1000-1 as described above.

The low elastic modulus region 1000-2 is a region capable of flexibly responding to an external force such as twisting, pressing, and pulling and may include, as described above, an elastomer having a relatively low elastic modulus, and accordingly, may provide the deformable section C of the stretchable display panel 2000 with stretchability to reduce stress acting when repetitively folded, bent, or rolled, and thus prevent or reduce damage in the deformable section C.

At least a portion of the thin film transistors electrically connected to the unit element 130 may be the aforementioned stretchable thin film transistor 300, and the stretchable thin film transistor 300 may be arranged in the high elastic modulus region 1000-1 or the low elastic modulus region 1000-2.

For example, the stretchable thin film transistor 300 may be arranged in the low elastic modulus region 1000-2, and thus, compared to a structure in which all thin film transistors are arranged in each pixel PX, the area occupied by the thin film transistor in the pixel PX may be reduced, thereby overcoming the space limitation of the pixels due to the low elastic modulus region 1000-2 for stretching, reducing the pixel size, and ultimately increasing the number of pixels per unit area. Accordingly, the deformable section C of the stretchable display panel 2000 may realize substantially the same resolution in the non-deformable section NC, and resultantly, uniform display quality over the entire screen may be achieved without deteriorating image quality in the deformable section C such as a bending section.

Unlike the deformable section C, the non-deformable section NC of the stretchable display panel 2000 may not include a separate low elastic modulus region 1000-2 and may include the high elastic modulus region 1000-1. Accordingly, the non-deformable section NC of the stretchable display panel 2000 may be covered with the non-stretchable pattern 110b on the stretchable substrate 110a, and the whole non-deformable section NC may be covered with, for example, a plate-shaped non-stretchable pattern 110b.

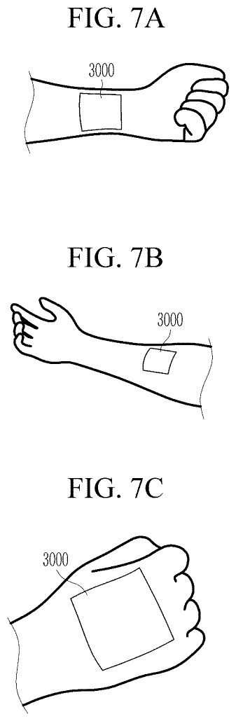

FIGS. 7A, 7B, and 7C are schematic views showing sensor arrays according to some examples.

Referring to FIGS. 7A through 7C, the sensor array 3000 according to some examples may be an attachable biometric sensor array, and may include the aforementioned stretchable panel 1000. The sensor array 3000 may be attached to a biological surface such as skin, a living body such as an organ, or an indirect means contacting a living body such as clothes to sense and measure biological information such as a biological signal. For example, the biometric sensor array includes an electroencephalogram (EEG) sensor, an electrocardiogram (ECG) sensor, a blood pressure (BP) sensor, an electromyography (EMG) sensor, a blood glucose (BG) sensor, a photoplethysmography (PPG) sensor, an accelerometer, a RFID antenna, an inertial sensor, an activity sensor, a strain sensor, a motion sensor, and/or any combination thereof, but is not limited thereto.

The sensor array 3000 may be attached to a living body in a very thin patch type or band type to monitor biometric information in real time. For example, the skin-type sensor array 3000 may be a sensor array including a photoplethysmography sensor (PPG sensor), and the biometric information may include heart rate, oxygen saturation, stress, arrhythmia, blood pressure, etc., and biometric information may be obtained by analyzing the waveform of an electrical signal.

The aforementioned stretchable panel 1000 and the stretchable display panel 2000 or the sensor array 3000 including the stretchable panel 1000 may be included in various electronic devices, and the electronic device may further include a processor (not shown) and a memory (not shown).

The electronic devices may include, for example, mobile phones, video phones, smart phones, smart pads, smart watches, digital cameras, tablet PCs, laptop PCs, notebook computers, computer monitors, wearable computers, televisions, digital broadcasting terminals, e-books, personal digital assistants (PDAs), PMP (portable multimedia player), EDA (enterprise digital assistant), head mounted displays (HMD), in-vehicle navigations, Internet of Things (IoT), Internet of Everything (IoE), security devices, and medical devices, but are not limited thereto. The operations and/or timing of the electronic device, including signals provided to the stretchable panel 1000, the stretchable display panel 2000, and/or the sensor array 3000 may be implemented using processing circuitry such as hardware, software, and/or a combination of hardware and software. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc. In some embodiments, the processing circuitry may include electrical components such as at least one of transistors, resistors, capacitors, etc., and/or electronic circuits including said components. For example, the processing circuitry may include electrical components such as logic gates including at least one of AND gates, OR gates, NAND gates, NOT gates, etc., and/or electronic circuits including said components.

Hereinafter, the embodiments are illustrated in more detail with reference to examples. However, these examples are exemplary, and the present scope is not limited thereto.

### EXAMPLES

### Preparation of Two-Dimensional Semiconductor Materials

### Synthesis Example 1: Synthesis of Acceptor Compound

As shown in Reaction Scheme 1, first, a side chain precursor in which an aliphatic alkyl group and an ethylene glycol (EG) structure are bonded in a branched form is synthesized as a branched side chain structure to be bonded to each nitrogen atom of a diketopyrrolopyrrole (DPP) moiety.

After the synthesis of the above side chain precursor, the synthesized side chain precursor is reacted with a compound in which a bromothiophene group is bonded to each pyrrole ring of DPP, thereby synthesizing an acceptor compound in which a side chain in which an aliphatic alkyl group and an EG group are branched from the synthesized side chain precursor are bonded to each nitrogen atom of DPP, and a bromothiophene is bonded to each pyrrole ring of DPP.

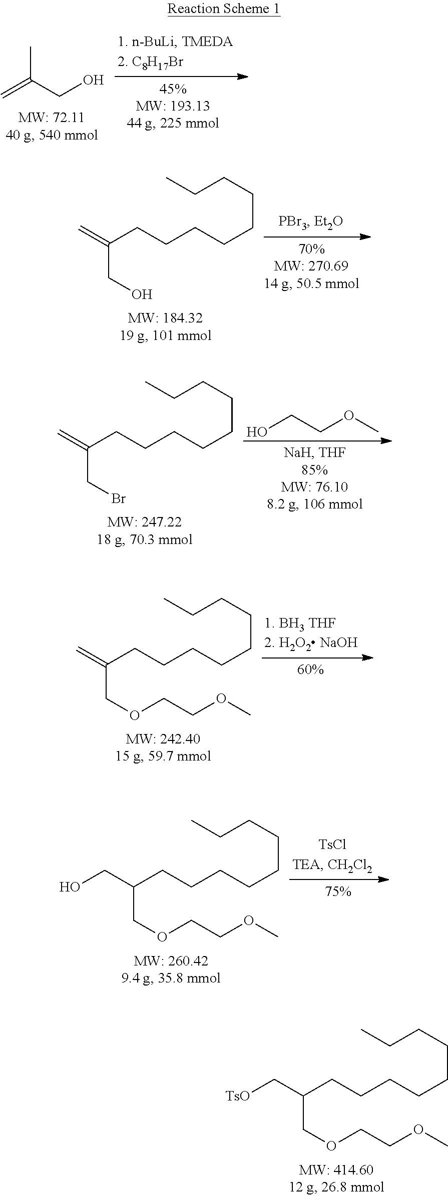

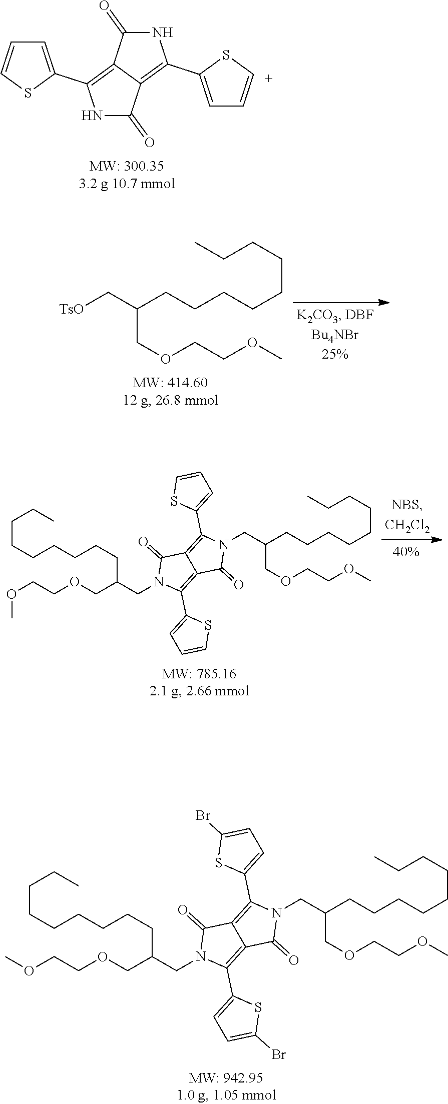

Specifically, 40 g (540 mmol) of 2-Methyl-2-propen-1-ol was dissolved in 500 ml of N,N,N′,N′-Tetramethyl ethylenediamine (TMEDA), cooled to −78° C., n-Butyllithium solution was added dropwise, and stirred for 1 hour. 44 g (225 mmol) of 1-bromooctane was added to the solution, the temperature was raised to room temperature, and the mixture was stirred for an additional 12 hours. After removing the solvent, the obtained product was separated and purified by silica gel column chromatography to obtain 19 g of 2-methylideneundecan-1-ol. The yield was 45%.

19 g (101 mmol) of 2-methylideneundecan-1-ol was dissolved in 200 ml of diethyl ether, and stirred at room temperature. 14 g (50.5 mmol) of phosphorus tribromide was added to the solution, and stirred for additional 12 hours at room temperature. After removing the solvent, the obtained product is separated and purified by silica gel column chromatography to obtain 18 g of 2-(bromomethyl) undec-1-ene. The yield is 70%.

Using the synthesized 2-(bromomethyl) undec-1-ene, the acceptor compound described at the end of Reaction Scheme 1 was synthesized using the same method as described in the literature (Angew. Chem. 2016, 128, 10532-10536).

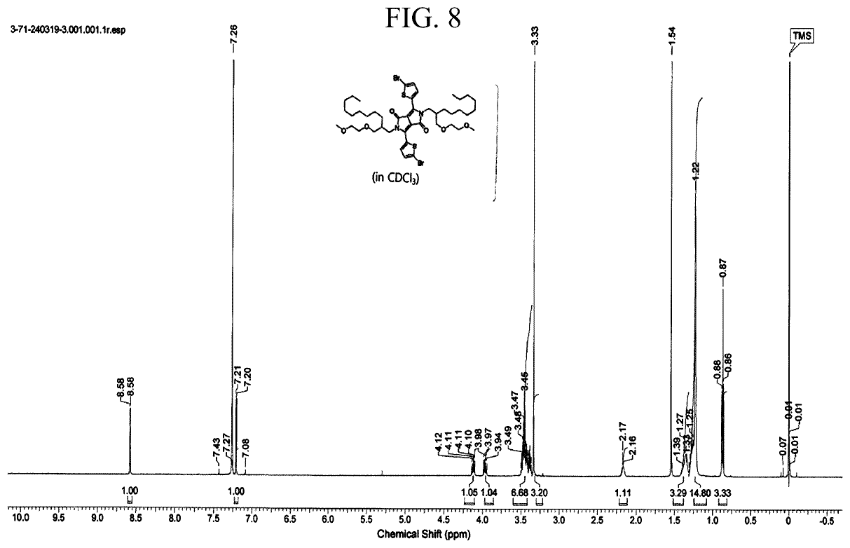

The final acceptor compound according to Reaction Scheme 1 was structurally analyzed using ¹H-NMR, and the results are shown in FIG. 8. ¹H-NMR spectra were measured at room temperature using a Bruker Avance III 600 (600 MHz) instrument in chloroform-d (CDCl₃) as the solvent.

### Example 1: Synthesis of Organic Semiconductor Polymer

As shown in Reaction Scheme 2, an organic semiconductor polymer according to Example 1 was synthesized by reacting the acceptor compound prepared in Synthesis Example 1 with 2,5-Bis(trimethylstannyl)-thieno[3,2-b]thiophene as a donor compound.

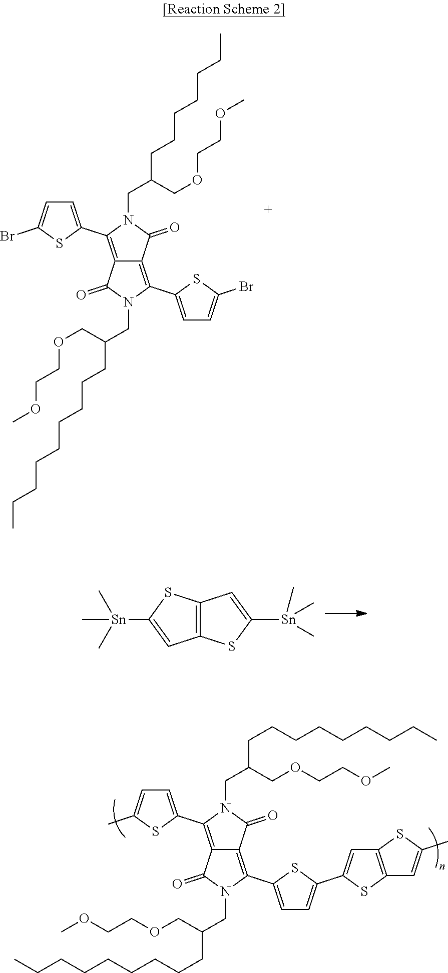

Specifically, 150 mg of the acceptor compound prepared in Synthesis Example 1, 74.1 mg of the donor compound (2,5-Bis(trimethylstannyl)-thieno[3,2-b]thiophene), 2.9 mg of Tris(dibenzylideneacetone)dipalladium(0) Pd₂(dba)₃, and 3.9 mg of Tri (o-tolyl) phosphine P (o-tolyl)3 are dissolved in 10 ml of anhydrous chlorobenzene (PhCl), and after nitrogen replacement, stirred at 130° C. for 24 hours. After cooling to room temperature, the solution is poured into 200 ml of methanol, and the resulting precipitate is filtered under reduced pressure to obtain the product (polymer). The resulting polymer is separated and purified in the order of acetone, n-hexane, methylene chloride, and chloroform using a Soxhlet apparatus, and the polymer solution dissolved in chloroform is distilled under reduced pressure to obtain the polymer. The yield obtained after drying in a vacuum for 24 hours is 100 mg.

### Example 2: Synthesis of Organic Semiconductor Polymer

As shown in Reaction Scheme 3, an organic semiconductor polymer according to Example 2 was synthesized using the same method as Example 1, except that 2,5-Bis(trimethylstannyl)thiophene, instead of 2,5-Bis(trimethylstannyl)-thieno[3,2-b]thiophene, was used as a donor compound and reacted with the acceptor compound prepared in Synthesis Example 1.

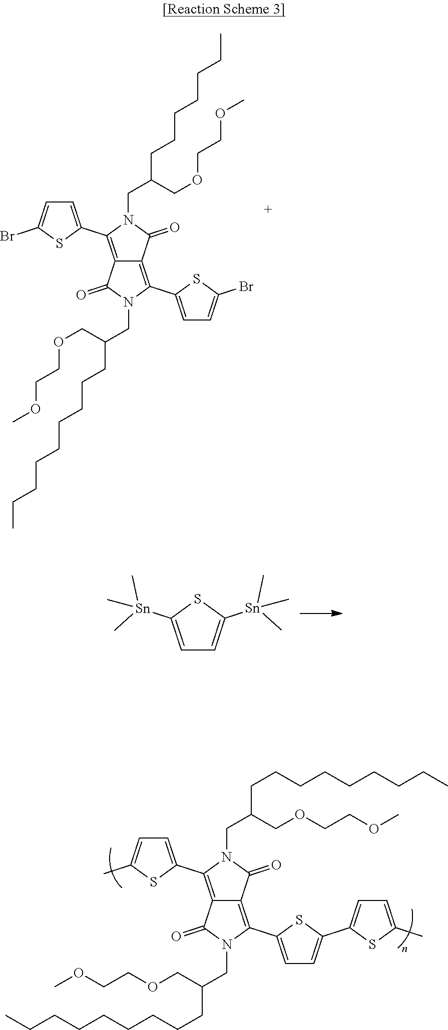

### Example 3: Synthesis of Organic Semiconductor Polymer

As shown in Reaction Scheme 4, an organic semiconductor polymer according to Example 3 was synthesized using the same method as Example 1, except that 2,5-Bis(trimethylstannyl) selenophene, instead of 2,5-Bis(trimethylstannyl)-thieno[3,2-b]thiophene, was used as a donor compound and reacted with the acceptor compound prepared in Synthesis Example 1.

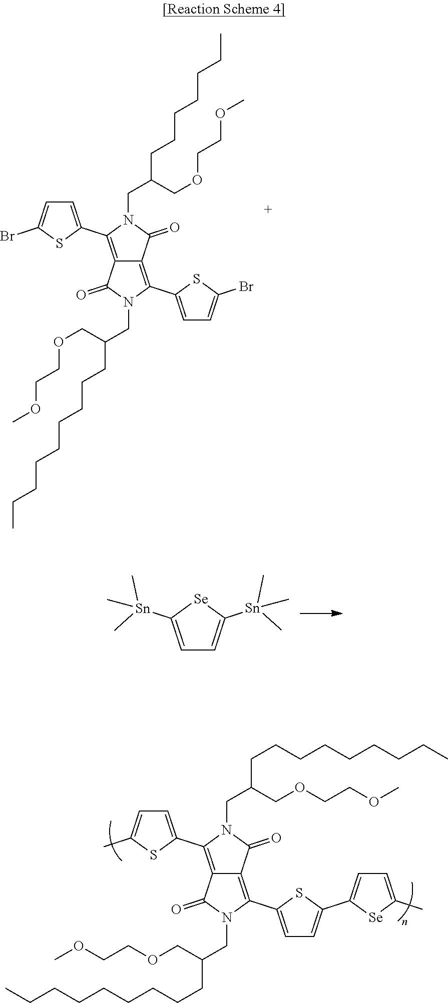

### Comparative Example 1: Synthesis of Organic Semiconductor Polymer

In the above Reaction Scheme 1, a side chain precursor containing two identical EG structures instead of an aliphatic alkyl group is synthesized as a branched side chain precursor, and this is reacted with a compound in which a bromothiophene group is each bonded to each pyrrole ring of DPP, thereby synthesizing an acceptor compound according to Comparative Synthesis Example 1 in which two branched EG structures derived from the synthesized side chain precursor are each bonded to a nitrogen atom of DPP, and a bromothiophene is bonded to each pyrrole ring of DPP.

The prepared acceptor compound of Comparative Synthesis Example 1 and the donor compound used in Example 3 are reacted in the same manner as in Example 3 to prepare an organic polymer according to Comparative Example 1, which is represented by the following chemical formula.

### Organic Polymer of Comparative Example 1

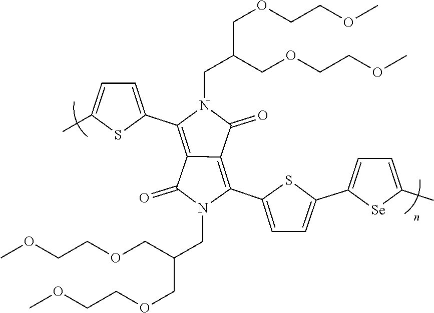

### Evaluation I

### (1) Light Absorbing Characteristics of Organic Polymer Thin Film

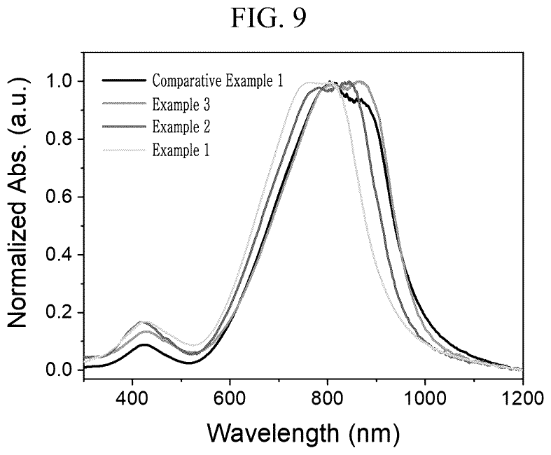

The organic polymers prepared in Examples 1 to 3 and Comparative Example 1 were each dissolved in chloroform at a concentration of 10 mg/ml to prepare organic polymer solutions, each of which was then spin-coated on a glass substrate (at room temperature, 1000 rpm to 2000 rpm for 60 seconds), dried at 180° C. for 1 hour to prepare a polymer thin film having a thickness of about 300 nm to about 500 nm. Optical absorption characteristics of the prepared thin films in the UV-Vis wavelength range were measured using a Varian Cary 500 Bio spectrophotometer, and the results are shown in FIG. 9.

As can be seen from FIG. 9, the organic polymers according to Examples 1 to 3 and Comparative Example 1 exhibit similar light absorption characteristics, although their side chain structures and donor structures are somewhat different from each other. In addition, by having branched side chain structures, all exhibited excellent solubility characteristics in solvents.

### (2) Surface Morphology of Organic Polymer Thin Film

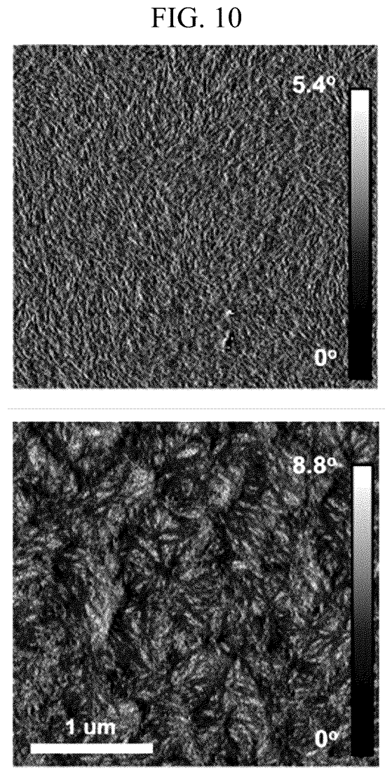

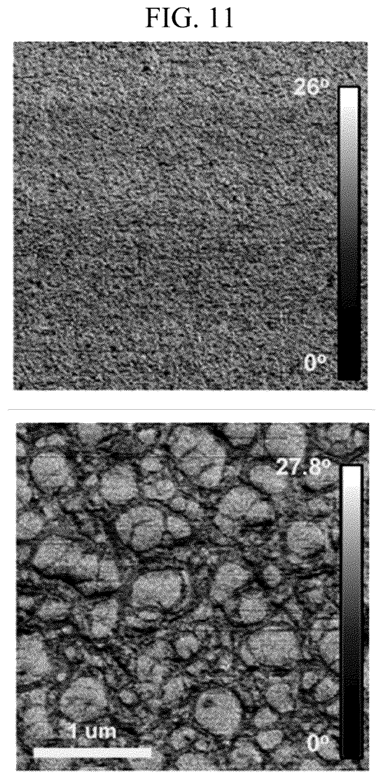

The surfaces of the organic polymer thin films prepared in Example 3 and Comparative Example 1 were measured using an atomic force microscope (AFM) device DimensionXR (manufactured by Dimension XR systems), and the results are shown in FIGS. 10 and 11, respectively.

FIG. 10 are images measuring the surface morphology of the organic polymer thin film according to Example 3. The upper image is a surface image of only the organic polymer thin film, and the lower image is an image measuring the surface of the organic polymer thin film coated on another polymer that can be used as a substrate of a stretchable transistor.

FIG. 11 are images measuring the surface morphology of the organic polymer thin film according to Comparative Example 1. The upper image is a surface image of only the organic polymer thin film, and the lower image is an image measuring the surface of the organic polymer thin film coated on another polymer that can be used as a substrate of a stretchable transistor.

As can be seen from FIG. 10, the surface of the organic polymer thin film according to Example 3 exhibits a uniform crystal structure, and the maximum height of the surface roughness is also relatively low.

On the other hand, as shown in FIG. 11, the surface of the organic polymer thin film according to Comparative Example 1 has an uneven shape including aggregated structures, and the surface roughness is also much higher than that of the organic polymer thin film according to Example 3. Although not intended to be bound by a specific theory, it can be inferred that this uneven surface morphology may be the cause of the device characteristics of the organic thin film transistor including the organic polymer according to Comparative Example 1, which will be described later, not being implemented at all.

### Manufacturing of Organic Thin Film Transistor

After 1000 Å of chromium used as a gate electrode is deposited on a cleaned glass substrate by sputtering, 1000 Å of SiO₂ used as a gate insulating film is deposited by CVD. Subsequently, 1200 Å of gold (Au) is deposited on the SiO₂ as a source-drain electrode by sputtering. Before depositing the organic semiconductor material, the substrate is washed with isopropyl alcohol for 10 minutes, dried, and used. Then, each of the organic polymers prepared in Examples 1 to 3 and Comparative Example 1 are dissolved in a 10 mg/ml concentration in an octadecyltrichlorosilane solution diluted in chloroform to a concentration of 10 mM, thereby preparing an organic semiconductor solution. The prepared solution is spin-coated at 1000 rpm on the substrate to a thickness of 1000 Å. Thereafter, the substrates are each baked at 100° C. for 1 hour under an argon atmosphere to fabricate organic thin film transistors each including the organic polymer according to Examples 1 to 3 and Comparative Example 1.

### Evaluation II

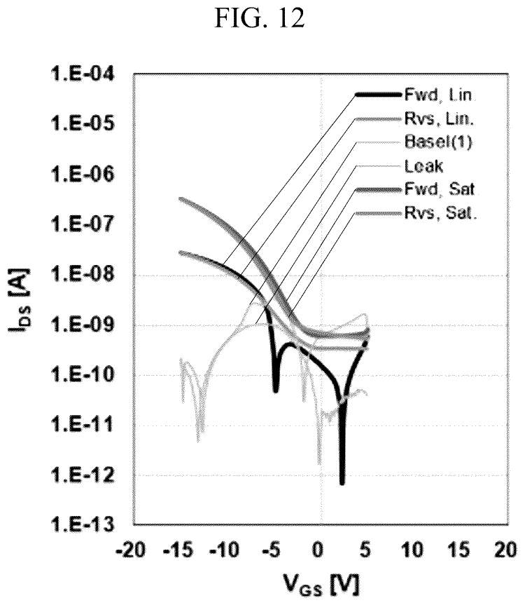

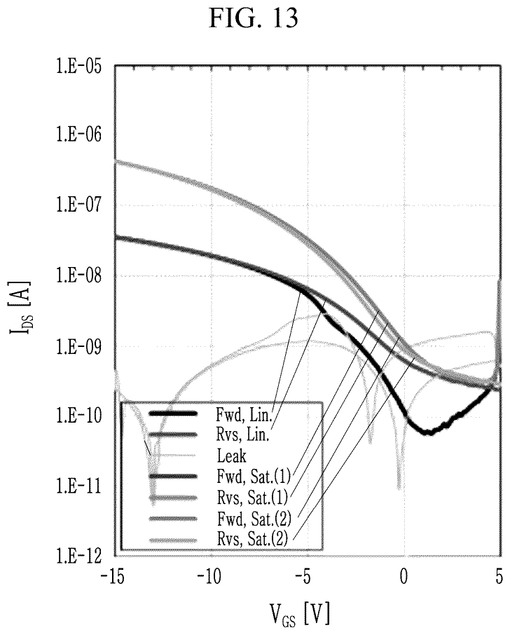

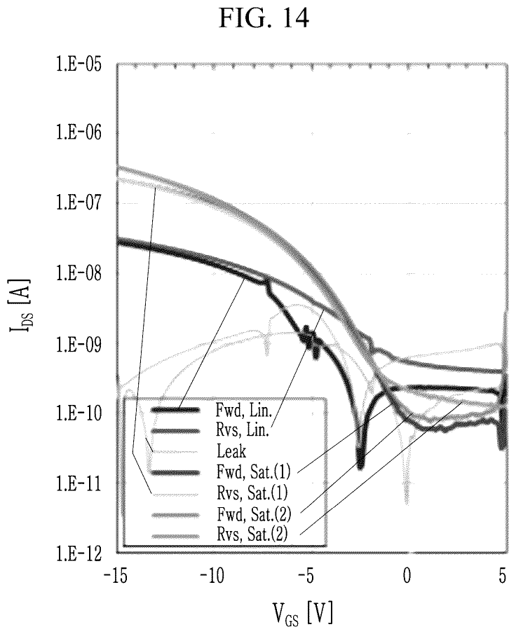

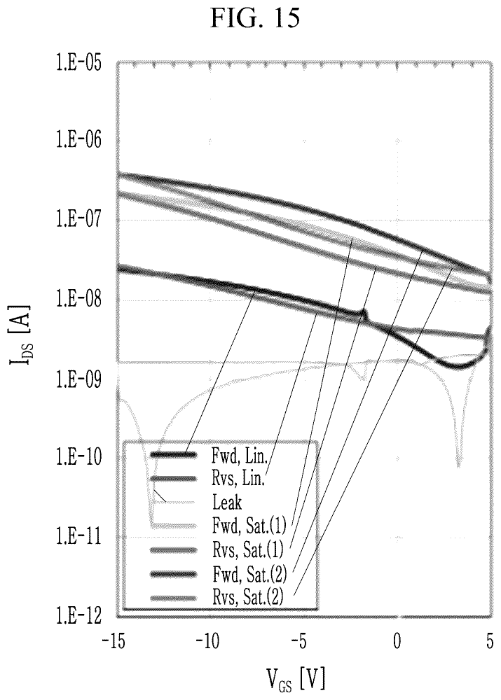

The transistor characteristics of the organic thin film transistors each including the organic polymers according to Examples 1 to 3 and Comparative Example 1 were measured, and the results are shown in FIGS. 12 to 15. In addition, from the results of FIGS. 12 to 15, the charge mobility, current on/off ratio (Iₒₙ/I_{off}), HOMO energy, optical band gap, and driving voltage of the organic thin film transistors each including the organic polymers according to Examples 1 to 3 and Comparative Example 1 were measured by the methods described below, and the results are shown in Table 1 below.

The charge mobility of the organic thin film transistor was obtained from the slope of a graph obtained from the saturation region current equation with (ISD)^{1/2} and V_{G} as variables.

$${I}_{SD} = \frac{{WC}_{0}}{2 L} {μ ( {V}_{G} - {V}_{T} )}^{2}$$

$$\sqrt{{I}_{SD}} = \sqrt{\frac{μ {C}_{0} W}{2 L}} ( {V}_{G} - {V}_{T} )$$

$$slope = \sqrt{\frac{μ {C}_{0} W}{2 L}}$$

$${μ}_{FET} = {( slope )}^{2} \frac{2 L}{{C}_{0} W}$$

In the above formulas, ISD is a source-drain current, μ or μ_{FET} is charge mobility, C₀ is electrostatic capacity of a gate insulating layer, W is a channel width, L is a channel length, V_{G} is a gate voltage, and VT is a threshold voltage.

A cut-off leakage current (I_{off}) is obtained as a minimum current in an off state as a current flowing in an off state. A current on-off ratio (Iₒₙ/I_{off}) is obtained as a ratio of a maximum current in an on state relative to a minimum current in the off state.

The results are in Table 1 below.

TABLE 1

| | | Optical | Hole | | |
|---|---|---|---|---|---|
| | HOMO | Bandgap | mobility | | |
| | (eV) | (eV) | (cm²/Vs) | Iₒₙ/I_{off} | Vₜₕ (V) |
| Comparative | 5.18 | 1.259 | N/A | N/A | N/A |
| Example 1 | | | | | |
| Example 1 | 5.03 | 1.310 | 0.05 | 5.04 × 10² | −5.02 |
| Example 2 | 5.11 | 1.280 | 0.05 | 1.12 × 10³ | −2.18 |
| Example 3 | 5.06 | 1.258 | 0.04 | 4.50 × 10³ | −3.06 |

Referring to FIGS. 12 to 15 and Table 1, it can be seen that the organic thin film transistors including the organic polymers according to Examples 1 to 3 exhibit excellent transistor characteristics, whereas the transistor including the organic polymer according to Comparative Example 1 does not exhibit charge mobility and does not implement transistor characteristics at all.

While this disclosure has been described in connection with what is presently considered to be some practical example embodiments, it is to be understood that the invention is not limited to the disclosed embodiments. On the contrary, it is intended to cover various modifications and equivalent arrangements included within the spirit and scope of the appended claims.

What is claimed is:

1. An organic semiconductor polymer comprising a structural unit represented by Chemical Formula 1: 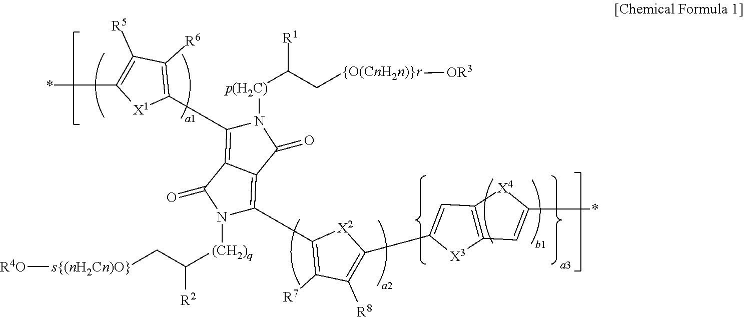
wherein in Chemical Formula 1,
R¹, R², R³, and R⁴ are each independently a substituted or unsubstituted C1 to C30 alkyl group,
R⁵, R⁶, R⁷, and R⁸ are each independently hydrogen, a substituted or unsubstituted C1 to C30 alkyl group, a substituted or unsubstituted C3 to C30 cycloalkyl group, a substituted or unsubstituted C6 to C30 aryl group, a substituted or unsubstituted C7 to C30 arylalkyl group, a substituted or unsubstituted C1 to C30 heteroalkyl group, a substituted or unsubstituted C2 to C30 heterocycloalkyl group, a substituted or unsubstituted C2 to C30 alkenyl group, a substituted or unsubstituted C2 to C30 alkynyl group, a hydroxy group, a halogen atom, or a combination thereof,
X¹, X², X³, and X⁴ are each independently S, Se, or Te,
a1 and a2 are each independently an integer from 1 to 5,
a3 is an integer from 0 to 5,
b1 is 0 or 1,
n is an integer from 2 to 5,
p and q are each independently an integer from 1 to 20, and
r and s are each independently an integer from 1 to 20.

2. The organic semiconductor polymer of claim 1, wherein
R¹, R², R³, and R⁴ are each independently a substituted or unsubstituted C1 to C20 alkyl group,
R⁵, R⁶, R⁷, and R⁸ are each independently hydrogen, a substituted or unsubstituted C1 to C20 alkyl group, a substituted or unsubstituted C3 to C20 cycloalkyl group, a substituted or unsubstituted C6 to C20 aryl group, a substituted or unsubstituted C7 to C20 arylalkyl group, a substituted or unsubstituted C1 to C20 heteroalkyl group, a substituted or unsubstituted C2 to C20 heterocycloalkyl group, a substituted or unsubstituted C2 to C20 alkenyl group, a substituted or unsubstituted C2 to C20 alkynyl group, a hydroxy group, a halogen atom, or a combination thereof,
X¹, X², X³, and X⁴ are each independently S or Se,
a1 and a2 are each independently from 1 to 3,
a3 is from 0 to 3,
b1 is 0 or 1,
n is an integer of 2 or 3,
p and q are each independently from 1 to 10, and
r and s are each independently from 1 to 10.

3. The organic semiconductor polymer of claim 1, wherein,
R¹ and R², are each independently a substituted or unsubstituted C5 to C20 alkyl group,
R³ and R⁴ are each independently a substituted or unsubstituted C1 to C5 alkyl group,
p and q are each independently from 2 to 10, and
r and s are each independently from 1 to 5.

4. The organic semiconductor polymer of claim 1, wherein,
R¹ and R², are each independently a substituted or unsubstituted C8 to C15 alkyl group,
R³ and R⁴ are each independently a substituted or unsubstituted C1 to C3 alkyl group,
p and q are each independently from 2 to 10, and
r and s are each independently from 1 to 5.

5. The organic semiconductor polymer of claim 1, wherein,
R⁵, R⁶, R⁷, and R⁸ are each independently hydrogen, a substituted or unsubstituted C1 to C10 alkyl group, a substituted or unsubstituted C3 to C10 cycloalkyl group, a substituted or unsubstituted C6 to C10 aryl group, a substituted or unsubstituted C7 to C10 arylalkyl group, a substituted or unsubstituted C1 to C10 heteroalkyl group, a substituted or unsubstituted C2 to C10 heterocycloalkyl group, a substituted or unsubstituted C2 to C10 alkenyl group, a substituted or unsubstituted C2 to C10 alkynyl group, a hydroxy group, a halogen atom, or a combination thereof,
X¹ and X² are each independently S, and
X³ and X⁴ are each independently S or Se.

6. The organic semiconductor polymer of claim 1, wherein,
a1, a2, and a3 are each independently 1 or 2, and
b1 is 0 or 1.

7. The organic semiconductor polymer of claim 1, wherein Chemical Formula 1 is represented by at least one of Chemical Formula 2, Chemical Formula 3, or Chemical Formula 4:

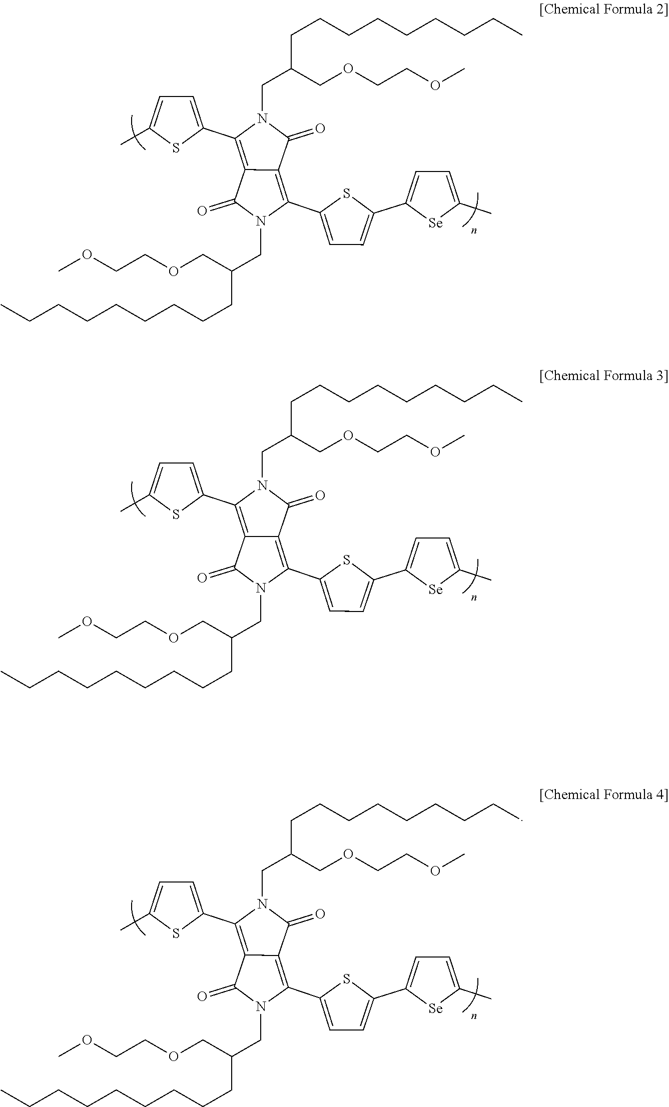

8. An organic thin film transistor comprising:
a gate electrode,
an organic semiconductor overlapping the gate electrode,
a gate insulating layer separating the gate electrode from the organic semiconductor; and
a source electrode and a drain electrode electrically connected to the organic semiconductor,
wherein the organic semiconductor comprises the organic semiconductor polymer of claim 1.

9. The organic thin film transistor of claim 8, wherein the organic thin film transistor is configured as a stretchable thin film transistor.

10. The organic thin film transistor of claim 9, wherein
the gate electrode, the source electrode, and the drain electrode each independently comprise at least one of a microcrack metal, a liquid metal, a conductive nanostructure, a conductive polymer, or a combination thereof, and
the gate insulating layer comprises at least one of polyorganosiloxane, a polymer including a butadiene structural unit, a polymer including an olefin structural unit, a polymer including a urethane structural unit, a polymer including an acrylic structural unit, or a combination thereof.

11. An electronic device comprising the organic thin film transistor of claim 9.

12. The electronic device of claim 11, wherein the electronic device comprises at least one of a display device or a sensor device.

13. An electronic device comprising an active layer, the active layer including the organic semiconductor polymer of claim 1.

14. The electronic device of claim 13, wherein the electronic device comprises at least one of a display device or a sensor device, and
the active layer is included in the at least one of the display device or the sensor device.

15. A stretchable panel comprising:
a stretchable substrate;
a stretchable thin film transistor array on the stretchable substrate, the stretchable thin film transistor array comprising a plurality of stretchable thin film transistors; and
a unit element array comprising a plurality of unit elements electrically connected to each of the plurality of stretchable thin film transistors,
wherein each of the plurality of stretchable thin film transistors comprises a gate electrode, an organic semiconductor overlapping with the gate electrode, and a source electrode and drain electrode each of which connected to the organic semiconductor, and
wherein the organic semiconductor comprises the organic semiconductor polymer of claim 1.

16. The stretchable panel of claim 15, further comprising
a non-stretchable pattern overlapping with a portion of the stretchable substrate, the non-stretchable pattern having a higher elastic modulus than the stretchable substrate,
wherein the stretchable panel comprises
a high elastic modulus region including the non-stretchable pattern, and
a low elastic modulus region excluding the high elastic modulus region.

17. The stretchable panel of claim 16, wherein
the stretchable substrate comprises at least one of a polyorganosiloxane, a polymer including a butadiene structural unit, a polymer including an olefin structural unit, a polymer including a urethane structural unit, a polymer including an acrylic structural unit, or a combination thereof, and
the non-stretchable pattern comprises at least one of a polycarbonate, a polymethylmethacrylate, a polyethylene terephthalate, a polyethylene naphthalate, a polyimide, a polyamide, a polyamideimide, a polyethersulfone, or a combination thereof.

18. The stretchable panel of claim 16, wherein each of the plurality of unit elements comprises at least one of a light emitting diode, a photoelectric conversion diode, or a combination thereof, and
the plurality of unit elements are in the high elastic modulus region.

19. The stretchable panel of claim 15, wherein the stretchable panel is at least one of a stretchable display or a stretchable sensor array.

20. An electronic device, comprising the stretchable panel of claim 15.
